SEQUENCE LISTING (8 sequences)

SEQ ID NO: 1 - protein, 127 aa, Artificial Sequence
  AB12 VH
QVQLVQSGAEVKKPGSSVKVSCKASGGTFSRYIINWVRQAPGQGLEWMGRIIPILGVENYAQKFQGRVTITADKSTSTAYMELSSLRSEDTAVYYCARKDWFDYWGQGTLVTVSSASTKGPSVFPLA

SEQ ID NO: 2 - protein, 119 aa, Artificial Sequence
  AB12 VL
EIVLTQSPGTLSLSPGERATLSCRASQSVSSYLAWYQQKPGQAPRLLIYGASSRATGIPDRFSGSGSGTDFTLTISRLEPEDFAVYYCQQYGSSPLTFGGGTKVEIKRTVAAPSVFIFP

SEQ ID NO: 3 - protein, 5 aa, Artificial Sequence
  VH CDR1
RYIIN

SEQ ID NO: 4 - protein, 17 aa, Artificial Sequence
  VH CDR2
RIIPILGVENYAQKFQG

SEQ ID NO: 5 - protein, 6 aa, Artificial Sequence
  VH CDR3
KDWFDY

SEQ ID NO: 6 - protein, 11 aa, Artificial Sequence
  VL CDR1
RASQSVSSYLA

SEQ ID NO: 7 - protein, 7 aa, Artificial Sequence
  VL CDR2
GASSRAT

SEQ ID NO: 8 - protein, 9 aa, Artificial Sequence
  VL CDR3
QQYGSSPLT

(19) United States
(12) Patent Application Publication
(10) Pub. No.: US 2022/0175948 A1
(43) Pub. Date: Jun. 9, 2022

(54) TREATMENT OF CAR T-CELL TOXICITY

(71) Applicants: ADC Therapeutics SA, Epalinges (CH); MedImmune Limited, Cambridge, Cambridgeshire (CH)

(72) Inventor: Tomasz Sablinski, Short Hills, NJ (US)

(21) Appl. No.: 16/968,143

(22) PCT Filed: Feb. 8, 2019
(86) PCT No.: PCT/EP2019/053165

§ 371 Date: Aug. 6, 2020

(30) Foreign Application Priority Data
Feb. 9, 2018 (GB) 1802172.5

(51) Int. Cl. A61K 47/68; A61P 35/00; A61K 45/06

(52) U.S. Cl. CPC A61K 47/6849; A61K 45/06; A61P 35/00

(57) ABSTRACT

The present disclosure relates to methods for improving the safety profile of adoptive cell transfer therapies. In particular, the present disclosure relates to methods for improving the safety profile of CAR T-cell therapies. Disclosed is an anti-CD25 antibody-drug-conjugate for use in a method of treatment or prevention of CAR immune cell toxicity in an individual.

### FIELD

The present disclosure relates to methods for improving the safety profile of adoptive cell transfer therapies. In particular, the present disclosure relates to methods for improving the safety profile of CAR T-cell therapies.

### BACKGROUND

Cell-Based Immunotherapy

Unlike surgery, radiotherapy, and chemotherapy, cell-based therapies can facilitate accurate decisions and execute highly complex behaviours [Tran E, et al., Science, 2014; 344:641-5//Gubin M M, et al., Nature, 2014; 515:577-81//Morrison C., Nat Biotechnol., 2015; 33:571-2]. Autologous lymphocytes isolated from a patient's own peripheral blood have the ability to specifically target tumor antigen and, once rendered capable of eliminating cancer cells expanded ex vivo, can be reinfused into the patient to attack any malignant tumor [Rosenberg S A, et al., Science. 2015; 348:62-8//Galluzzi L, et al., Oncoimmunology, 2012; 1:306-15//Kalos M, June C H., Immunity, 2013; 39:49-60; Restifo N P, et al., Nat Rev Immunol., 2012; 12:269-81]. The majority of preclinical and clinical data regarding autologous T-cells have highlighted the safety of using the cell therapy and the lack of potential for graft-versus-host disease (GvHD) mediated by the allogeneic T-cells [Mochizuki K, et al., Blood, 2016; 127:3270-80//Blazar B R, et al., Nat Rev Immunol., 2012; 12:443-58//Maude S L, et al., Blood, 2015; 125:4017-23].

T-cells antigen specificity is conferred by the T-cell receptor (TCR), an α/β heterodimer, which has the ability to redirect the tumor antigens in a major histocompatibility complex-dependent (MHC) manner [Rudolph M G, et al., Annu Rev Immunol., 2006, 24:419-66]. The T-cells are activated upon TCR combining with other cell surface molecules, termed TCR/CD3 complex, which contains ten immunoreceptor tyrosine activation motifs (ITAMs) and twenty tyrosine-phosphorylation sites [Cole D K, et al., J Immunol., 2007, 178:5727-34//Birnbaum M E, et al. PNAS USA, 2014, 111:17576-81]. However, the efficacy of T-cell treatments using conventional T-cells is limited by the so-called “tumor escape mechanism” through which tumour cells downregulate MHC expression on their surface. This effectively conceals the tumour cells from targeting by conventional T-cells, since interaction between T-cell receptor and peptide-MHC is a prerequisite for the activation of conventional T-cells [Dunn G P, et al., Nat Immunol., 2002, 3:991-8//Nagaraj S, Gabrilovich D I., Cancer Res., 2008, 68:2561-3//Beatty G L, Gladney W L., Clin Cancer Res., 2015, 21:687-92].

CAR T-Cells

One approach to overcome some of the limitations of conventional T-cells is to use in their place T cells that have been genetically engineered to express a chimeric antigen receptor (CAR) [FIG. 1A, Hartmann, J., et al., EMBO Mol. Med., 2017, Vol. 9, Issue 9, pp. 1183-1197]. Such CAR T-cells recognize surface antigens independently from MHC restriction. When targeted to tumor surface antigens, CAR T-cells proliferate and kill tumor cells upon antigen contact [Fesnak et al, 2016, Nat Rev Cancer 16: 566-581]).

CARs are composed of an extracellular binding domain, a hinge region, a transmembrane domain, and one or more intracellular signalling domains (FIG. 1B). Single-chain variable fragments (scFvs) derived from tumor antigen-reactive antibodies are commonly used as extracellular binding domains. All CARs harbour the CD3epsilon chain domain as the intracellular signalling domain. Second- or third-generation CARs also contain co-stimulatory domains, like CD28 and/or 4-1BB, improving proliferation, cytokine secretion, resistance to apoptosis, and in vivo persistence. Third-generation CARs exhibit improved effector functions and in vivo persistence as compared to second-generation CARs, whereas fourth-generation CARs, so-called TRUCKs (T-cells Redirected for Universal Cytokine Killing) or armoured CARs, combine the expression of a second-generation CAR with factors that enhance anti-tumoral activity, such as cytokines, costimulatory ligands, or enzymes that degrade the extracellular matrix of solid tumors [FIG. 1B; Chmielewski & Abken, 2015, Expert Opin Biol Ther 15, pp. 1145-1154].

Toxicity and management of side effects in CAR T-cell therapy CAR T-cells combine the abilities of high cell-killing power, self-amplification, and persistence in the individual once administered. This explains the remarkable efficacy that has been reported for CAR T-cells in a number of clinical trials (see NCT00968760, NCT01865617, NCT01815749, NCT01626495, and NCT01044069, where more than 85% of treated individuals reached complete response; nb. the ‘NCT numbers’ [NCTxxxxxxxx] represent unique identifiers for clinical trials, the details of which can be accessed by searching the https://clinicaltrials.gov/ct2/home website using the relevant NCT number). However, the same properties mean that toxic reactions to CAR T-cell treatment, when they occur, can be severe (see NCT02535364, NCT02348216, NCT02435849, NCT01865617, and NCT01044069; fatal toxicities occurred in all five trials, with significant levels of grade 3 or higher neurological toxicities in three of the five trials).

Several different classes of toxicity have been associated with CAR T-cell treatment. The most widely-reported toxicity of CAR T-cell therapy is cytokine release syndrome (CRS). The hallmark of CRS is immune activation resulting in elevated inflammatory cytokines especially IL-6 [Lee et al, 2014, Blood 124: 188-195]. Symptoms such as high fever, fatigue, nausea, tachycardia/hypotension, and cardiac dysfunction have most often been reported in trials with CD19-CARs but also occurred when other antigens of haematological malignancies, or mesothelin for the treatment of solid tumors, were targeted [Beatty et al, 2014, Cancer Immunol Res 2: 112-120]. Systemic corticosteroid administration rapidly reversed symptoms in most cases [Davila et al, 2014, Sci Transl Med 6: 224ra25//Lee et al, 2015, Lancet 385:517-528], but, can result in ablation of the infused CAR T cells, thus limiting the anti-tumoral effect [Davila et al, ibid.]. A currently preferred alternative is treatment with tocilizumab, a therapeutic IL-6 receptor blocking antibody, which does not affect CAR T cell persistence [Davila et al, ibid.; Maude et al, 2014, N Engl J Med 371: 1507-1517]. However, one death case has been reported due to severe CRS with multi-organ failure 3 days after the CAR-T cell infusion, despite treatment with tocilizumab, the TNFa inhibitor etanercept, and corticosteroids [Turtle et al, 2016, J Clin Investig 126: 2123-2138]. Nonetheless, FDA approval of the use of tocilizumab for treatment of CAR cell toxicity was announced on 30 Aug. 2017, the same day that FDA approval was given to the use of Kymriah (anti-CD19 CAR T-cells) in treating ALL.

Several deaths from neurotoxicity following CAR T-cell treatment have also been reported. For example, in the CD19-CAR T trial (NCT01865617) 122 days after CAR T cell infusion [Turtle et al, ibid.]. Furthermore, reversible symptoms of neurotoxicity including confusion, delirium, expressive aphasia, encephalopathy, and seizures were reported in several other studies [Brentjens et al, 2011, Sci Transl Med 5: 177ra38//Maude et al, ibid.//Kochenderfer et al, 2015, J Clin Oncol 33: 540-549//Lee et al, ibid.//Turtle et al, ibid.]. The potential causes for the neurotoxicity are unclear, with cytokine diffusion and/or translocation of activated CAR T-cell across the blood-brain barrier both postulated mechanisms.

Another safety concern associated with the CAR-T cell therapy is damage to healthy tissue due to the presence of the “tumor antigens” on the non-tumour tissues [Gross G, Eshhar Z., Annu Rev Pharmacol Toxicol. 2016, 56:59-83//Kershaw M H, et al., Nat Rev Cancer, 2013, 13:525-41]. Truly tumor-specific antigens appear rare, especially for solid tumors [Johnson L A, et al., Blood, 2009, 114:535-46]. Therefore, most targeting molecules for CARs are, in fact, “tumour associated” rather than “tumour specific” (i.e. they are expressed at higher levels on tumour cells, but appear at lower levels on some healthy tissues). The power of CAR T-cell therapy means that even these low levels of antigen can be sufficient to provoke destruction of the healthy tissue.

This potentially lethal toxicity, described as “on-target, off-tumor” effect, to date, has been reported in several CAR T-cell clinical trials. For example, in NCT00924326, CAR T-cells targeting the CD19 antigen enriched on B-cell malignancies have been reported to lead to long-term B-cell aplasia (targeting and eradication of normal B cells) [Kochenderfer J N, et al., Blood, 2012, 119:2709-20].

In order to improve the safety profile of CAR T-cell therapy, several studies have explored strategies for exerting enhanced levels of control over CAR T-cell therapy. These strategies include the use of suicide genes, inhibitory CARs, dual-antigen receptors, and exogenous molecules as switches to control the CAR-T cell functions [reviewed in Zhang and Xu, J. Hematology & Oncology, 2017, 10:1, DOI 10.1186/s13045-016-0379-6]. Enhanced control and safety features are expected to facilitate regulatory approval of CAR T-cell therapies, as well as acceptance of the treatments by clinicians and patients. Accordingly, there remains on ongoing need for the development of safe and effective mechanisms to regulate CAR T-cell therapies.

### SUMMARY

Realising that the principal cause of CAR T-cell toxicity is the uncontrolled and excessive activation and proliferation of CAR T-cells, the present authors reasoned that CAR T-cell toxicity could be treated by reducing CAR T-cell numbers, in particular the number of activated CAR T-cells. The present authors deduced that this reduction in CAR T-cell number could be achieved in a targeted and effective manner thought the administration of an antibody drug conjugate (ADC) targeted to CD25 (IL-2 receptor), a surface antigen present on T-cells which is upregulated upon T-cell activation.

Accordingly, in one aspect the present disclosure provides a method for treating or preventing CAR immune cell toxicity in an individual, the method comprising administering to the individual an effective amount of an ADC.

In a second aspect, the present disclosure provides an ADC for use in a method of treatment or prevention of CAR immune cell toxicity in an individual, wherein the treatment comprises administration of the ADC to the individual.

In a third aspect, the present disclosure provides a pharmaceutical composition comprising an ADC for use in a method of treating or preventing CAR immune cell toxicity in an individual, wherein the treatment comprises administration of the ADC to the individual. The composition may comprise a pharmaceutically acceptable excipient

In a fourth aspect, the present disclosure provides a use of an ADC in the manufacture of a medicament for treating or preventing CAR immune cell toxicity in an individual, wherein the medicament comprises an ADC, and wherein the treatment comprises administration of the medicament to the individual.

In a fifth aspect, the present disclosure provides a kit comprising a first medicament comprising an ADC and a package insert comprising instructions for administration of the first medicament to an individual for the treatment or prevention of CAR immune cell toxicity.

Typically, a CAR immune cell has been administered to the individual prior to administration of the ADC. The CAR immune cell may have been administered to the individual at least 1 hour, 2 hours, 4 hours, 8 hours, 24 hours, 48 hours, 72 hours, 1 week, 2 weeks, 4 weeks, 3 months, 6 months, 12 months, 2 years, or 5 years prior to administration of the ADC.

The CAR immune cell may express a CAR that specifically binds a tumour associated antigen. The CAR may specifically bind CD19, CD20, BCMA, CD22 or may bind to a solid tumor target.

Preferably, the CAR immune cell is a CAR T-cell. The CAR T-cell may be a 1^{st} generation CAR T-cell, a 2^{nd} generation CAR T-cell, a 3^{rd} generation CAR T-cell, a 4^{th} generation CAR T-cell, a TRUCK, a smart CAR, or an iCAR.

The individual may have CAR immune cell toxicity or have been determined to have CAR immune cell toxicity. The individual may not have CAR immune cell toxicity or may not have been determined to have CAR immune cell toxicity.

The individual may have had a disorder, or may have been determined to have had a disorder, wherein the individual has reached complete response (CR) to the disorder or has been determined to have reached complete response (CR) to the disorder. The disorder may have been a proliferative disease, such as cancer or a haematological cancer. The disorder may have been selected from the group comprising: Hodgkin's and non-Hodgkin's Lymphoma, including diffuse large B-cell lymphoma (DLBCL), follicular lymphoma, (FL), Mantle Cell lymphoma (MCL), chronic lymphatic lymphoma (CLL), Marginal Zone B-cell lymphoma (MZBL) and leukemias such as Hairy cell leukemia (HCL), Hairy cell leukemia variant (HCL-v), Acute Myeloid Leukaemia (AML), and Acute Lymphoblastic Leukaemia (ALL) such as Philadelphia chromosome-positive ALL (Ph+ALL) or Philadelphia chromosome-negative ALL (Ph−ALL).

The ADC may be administered in a dosage regime sufficient to eliminate or inactivate CAR immune cells in the individual. The elimination of CAR T-cells may be assesses by the absence of detectable CAR T-cells in a sample taken from the individual.

The ADC may be administered as a single dose. The ADC may be administered as two doses. The second dose may be administered 1 week after the first dose, or 3 weeks after the first dose. Each dose may be about 10, 20, 30, 40, 50, 60, 70, 80, 90, 100, 120, 150, 200, 250, or 300 μg/kg of ADC. Each dose may be about 1 to 10 μg/kg, 11 to 20 μg/kg, 21 to 30 μg/kg, 31 to 40 μg/kg, 41 to 50 μg/kg, 51 to 60 μg/kg, 61 to 70 μg/kg, 71 to 80 μg/kg, 81 to 90 μg/kg, 91 to 100 μg/kg, 101 to 120 μg/kg, 121 to 140 μg/kg, 141 to 160 μg/kg, 161 to 180 μg/kg, 181 to 200 μg/kg, 201 to 220 μg/kg, 221 to 240 μg/kg, 241 to 260 μg/kg, 261 to 280 μg/kg, or 281 to 300 μg/kg ADC.

The individual may be human.

The CAR immune cell toxicity may be one or more disorder selected from the following:
(a) Cytokine Release syndrome (CRS), such as elevated IL-6, elevated interferon gamma, elevated tumour necrosis factor, pyrexia, fatigue, nausea, tachycardia, hypotension, dyspnea, shortness of breath, pulmonary edema, or cardiac dysfunction;
(b) Neurotoxicity, such as cerebral oedema, confusion, delirium, aphasia, or encephalopathy;
(c) Tumour Lysis Syndrome (TLS), such as hyperuricemia, or hyperkalemia;
(d) Cellular and/or humoral immune responses, such as anaphylaxis;
(e) On-target, off-tumour recognition; or
(f) Off-target, off-tumour recognition.

Preferably the CAR immune cell toxicity is CAR T-cell toxicity.

The symptoms of on-target, off-tumour recognition depends on the antigen targeted by the CAR immune cell. In individuals treated with CAR immune cells targeting B-cell malignancies (such as anti-CD19 CAR immune cells)), on-target, off-tumour toxicity may manifest as B-cell aplasia.

The ADC may be an anti-CD25 ADC. Preferably, the anti-CD25 ADC is ADCX25, ADCT-301, or Camidanlumab tesirine.

The ADC may be administered in combination with a second therapeutic agent. The second therapeutic agent may be a corticosteroid such as dexamethasone, or an IL-6 antagonist such as tocilizumab.

### DETAILED DESCRIPTION

Antibody Therapy

Antibody therapy has been established for the targeted treatment of subjects with cancer, immunological and angiogenic disorders (Carter, P. (2006) Nature Reviews Immunology 6:343-357). Suitable target cells include immune cells such as T-cells, which are the target in T-cell lymphomas such as ALL (described in more detail below). The use of antibody-drug conjugates (ADC), i.e. immunoconjugates, for the local delivery of cytotoxic or cytostatic agents, i.e. drugs to kill or inhibit tumour cells in the treatment of cancer, targets delivery of the drug moiety to tumours, and intracellular accumulation therein, whereas systemic administration of these unconjugated drug agents may result in unacceptable levels of toxicity to normal cells (Xie et al (2006) Expert. Opin. Biol. Ther. 6(3):281-291; Kovtun et al (2006) Cancer Res. 66(6):3214-3121; Law et al (2006) Cancer Res. 66(4):2328-2337; Wu et al (2005) Nature Biotech. 23(9):1137-1145; Lambert J. (2005) Current Opin. in Pharmacol. 5:543-549; Hamann P. (2005) Expert Opin. Ther. Patents 15(9):1087-1103; Payne, G. (2003) Cancer Cell 3:207-212; Trail et al (2003) Cancer Immunol. Immunother. 52:328-337; Syrigos and Epenetos (1999) Anticancer Research 19:605-614).

CD25

The type I transmembrane protein CD25 is present on activated T- and B-cells, some thymocytes, myeloid precursors, and oligodendrocytes. Upon activation, surface expression of CD25 on T-cells is upregulated (Kmieciak et al., 2009, J. Trans. Med., 7:89). On activated T-cells, it forms heterodimers with the beta- and gamma subunits (CD122 and CD132), thus comprising the high-affinity receptor for IL-2. This ligand represents a survival factor for activated T-cells, as removal of IL-2 leads to immediate death of these cells.

In case of B-cells, CD25 is physiologically expressed in early developmental stages of late pro-B and pre-B cells. Malignancies arising from this stage of B-cell differentiation may thus also express CD25. Mast cell lesions are also positive for CD25 which is thus considered as a key diagnostic criterion for determination of systemic mastocytosis. In Hodgkin lymphomas, CD25 is reported to be not expressed in Hodgkin-/Reed-Sternberg cells in nodular lymphocyte predominance Hodgkin lymphoma (NLPHL), whereas the same cell type expresses CD25 at varying levels in classical Hodgkin' lymphomas of mixed cellularity type. The general expression levels are reported to be lower than in tumor infiltrating lymphocytes (TILs), which may result in problems demonstrating CD25 tumor cells in these cases (Merz et al, 1995, Lab Invest. 1995 July; 73(1):149-56).

Expression of the target antigen has also been reported for several B- and T-cell-derived subtypes of non-Hodgkin-lymphomas, i.e. B-cell chronic lymphatic leukemia, hairy cell leukemia, small cell lymphocytic lymphoma/chronic lymphocytic leukemia as well as adult T-cell leukemia/lymphoma and anaplastic large cell lymphoma.

CD25 may be localised to the membrane, with some expression observed in the cytoplasm. Soluble CD25 may also be observed outside of cells, such as in serum.

Therapeutic Uses of Anti-CD25 ADCs

The efficacy of an Antibody Drug Conjugate comprising an anti-CD25 antibody (an anti-CD25-ADC) in the treatment of, for example, cancer has been established—see, for example, WO2014/057119, WO2016/083468, and WO2016/166341.

Antibody Drug Conjugates (ADCs)

The present disclosure relates to the improved efficacy of combinations of an ADC and a secondary agent.

The ADC can deliver a drug to a target location. The target location is preferably a proliferative cell population. The antibody is an antibody for an antigen present on a proliferative cell population. In one aspect the antigen is absent or present at a reduced level in a non-proliferative cell population compared to the amount of antigen present in the proliferative cell population, for example a tumour cell population.

The ADC may comprise a linker which may be cleaved so as to release the drug at the target location. The drug may be a compound selected from RelA, RelB, RelC, RelD or RelE. Thus, the conjugate may be used to selectively provide a compound RelA, RelB, Rel C, RelD or RelE to the target location.

The linker may be cleaved by an enzyme present at the target location.

The disclosure particularly relates treatment with an anti-CD25 ADC disclosed in WO2014/057119, and as herein described.

Anti-CD25 ADCs

As used herein, the term “CD25-ADC” refers to an ADC in which the antibody component is an anti-CD25 antibody. The term “PBD-ADC” refers to an ADC in which the drug component is a pyrrolobenzodiazepine (PBD) warhead. The term “anti-CD25-ADC” refers to an ADC in which the antibody component is an anti-CD25 antibody, and the drug component is a PBD warhead.

The ADC may comprise a conjugate of formula L-(DL)ₚ, where DL is of formula I or II:

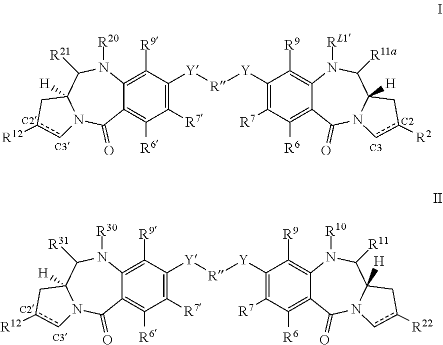

wherein:

L is an antibody (Ab) which is an antibody that binds to CD25;
when there is a double bond present between C2′ and C3′, R¹² is selected from the group consisting of:

(ia) C₅₋₁₀ aryl group, optionally substituted by one or more substituents selected from the group comprising: halo, nitro, cyano, ether, carboxy, ester, C₁₋₇ alkyl, C₃₋₇ heterocyclyl and bis-oxy-C₁₋₃ alkylene;

(ib) C₁₋₅ saturated aliphatic alkyl;

(ic) C₃₋₆ saturated cycloalkyl;

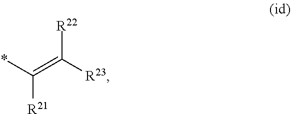

wherein each of R²¹, R²² and R²³ are independently selected from H, C₁₋₃ saturated alkyl, C₂₋₃ alkenyl, C₂₋₃ alkynyl and cyclopropyl, where the total number of carbon atoms in the R¹² group is no more than 5;

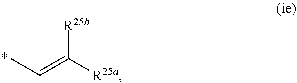

wherein one of R^{25a} and R^{25b} is H and the other is selected from: phenyl, which phenyl is optionally substituted by a group selected from halo, methyl, methoxy; pyridyl; and thiophenyl; and

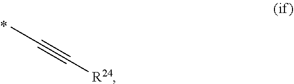

where R²⁴ is selected from: H; C₁₋₃ saturated alkyl; C₂₋₃ alkenyl; C₂₋₃ alkynyl; cyclopropyl; phenyl, which phenyl is optionally substituted by a group selected from halo, methyl, methoxy; pyridyl; and thiophenyl;

when there is a single bond present between C2′ and C3′,

R¹² is

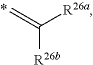

where R^{26a} and R^{26b} are independently selected from H, F, C₁₋₄ saturated alkyl, C₂₋₃ alkenyl, which alkyl and alkenyl groups are optionally substituted by a group selected from C₁₋₄ alkyl amido and C₁₋₄ alkyl ester; or, when one of R^{26a} and R^{26b} is H, the other is selected from nitrile and a C₁₋₄ alkyl ester;

R⁶ and R⁹ are independently selected from H, R, OH, OR, SH, SR, NH₂, NHR, NRR′, nitro, Me₃Sn and halo;

where R and R′ are independently selected from optionally substituted C₁₋₁₂ alkyl, C₃₋₂₀ heterocyclyl and C₆₋₂₀ aryl groups;

R⁷ is selected from H, R, OH, OR, SH, SR, NH₂, NHR, NHRR′, nitro, Me₃Sn and halo;

R″ is a C₃₋₁₂ alkylene group, which chain may be interrupted by one or more heteroatoms, e.g. O, S, NR^{N2} (where R^{N2} is H or C₁₋₄ alkyl), and/or aromatic rings, e.g. benzene or pyridine;

Y and Y′ are selected from O, S, or NH;

R^{6′}, R^{7′}, R^{9′} are selected from the same groups as R⁶, R⁷ and R⁹ respectively;

[Formula I]

R^{L1′} is a linker for connection to the antibody (Ab);

R^{11a} is selected from OH, OR^{A}, where R^{A} is C₁₋₄ alkyl, and SO_{z}M, where z is 2 or 3 and M is a monovalent pharmaceutically acceptable cation;

R²⁰ and R²¹ either together form a double bond between the nitrogen and carbon atoms to which they are bound or;

R²⁰ is selected from H and R^{C}, where R^{C} is a capping group;

R²¹ is selected from OH, OR^{A} and SO_{z}M;

when there is a double bond present between C2 and C3, R² is selected from the group consisting of:

(ia) C₅₋₁₀ aryl group, optionally substituted by one or more substituents selected from the group comprising: halo, nitro, cyano, ether, carboxy, ester, C₁₋₇ alkyl, C₃₋₇ heterocyclyl and bis-oxy-C₁₋₃ alkylene;

(ib) C₁₋₅ saturated aliphatic alkyl;

(ic) C₃₋₆ saturated cycloalkyl;

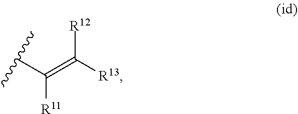

wherein each of R¹¹, R¹² and R¹³ are independently selected from H, C₁₋₃ saturated alkyl, C₂₋₃ alkenyl, C₂₋₃ alkynyl and cyclopropyl, where the total number of carbon atoms in the R² group is no more than 5;

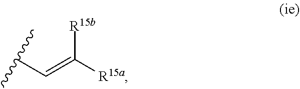

wherein one of R^{15a} and R^{15b} is H and the other is selected from: phenyl, which phenyl is optionally substituted by a group selected from halo, methyl, methoxy; pyridyl; and thiophenyl; and

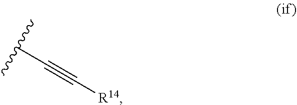

where R¹⁴ is selected from: H; C₁₋₃ saturated alkyl; C₂₋₃ alkenyl; C₂₋₃ alkynyl; cyclopropyl; phenyl, which phenyl is optionally substituted by a group selected from halo, methyl, methoxy; pyridyl; and thiophenyl;

when there is a single bond present between C2 and C3,

R² is

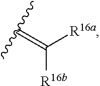

where R^{16a} and R^{16b} are independently selected from H, F, C₁₋₄ saturated alkyl, C₂₋₃ alkenyl, which alkyl and alkenyl groups are optionally substituted by a group selected from C₁₋₄ alkyl amido and C₁₋₄ alkyl ester; or, when one of R^{16a} and R^{16b} is H, the other is selected from nitrile and a C₁₋₄ alkyl ester;

[Formula II]

R²² is of formula IIIa, formula IIIb or formula IIIc:

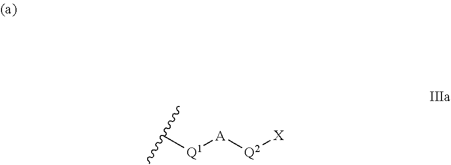

where A is a C₅₋₇ aryl group, and either

(i) Q¹ is a single bond, and Q² is selected from a single bond and —Z—(CH₂)ₙ—, where Z is selected from a single bond, O, S and NH and n is from 1 to 3; or

(ii) Q¹ is —CH═CH—, and Q² is a single bond;

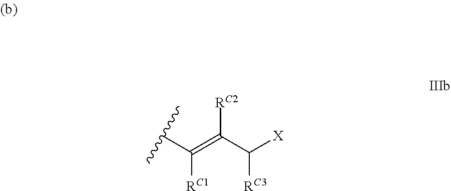

where;

R^{C1}, R^{C2} and R^{C3} are independently selected from H and unsubstituted C₁₋₂ alkyl;

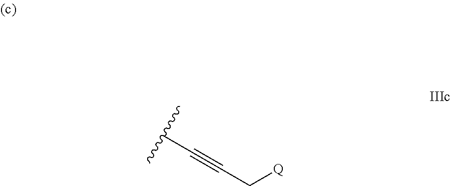

where Q is selected from O—R^{L2′}, S—R^{L2′} and NR^{N}—R^{L2′}, and R^{N} is selected from H, methyl and ethyl

X is selected from the group comprising: O—R^{L2′}, S—R^{L2′}, CO₂—R^{L2′}, CO—R^{L2′}, NH—C(═O)—R^{L2′}, NHNH—R^{L2′}, CONHNH—R^{L2′},

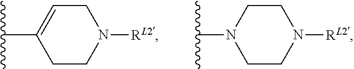

NR^{N}R^{L2′}, wherein R^{N} is selected from the group comprising H and C₁₋₄ alkyl;

R^{L2′} is a linker for connection to the antibody (Ab);

R¹⁰ and R¹¹ either together form a double bond between the nitrogen and carbon atoms to which they are bound or;

R¹⁰ is H and R¹¹ is selected from OH, OR^{A} and SO_{z}M;

R³⁰ and R³¹ either together form a double bond between the nitrogen and carbon atoms to which they are bound or;

R³⁰ is H and R³¹ is selected from OH, OR^{A} and SO_{z}M.

In some embodiments L-R^{L1′} or L-R^{L2′} is a group:

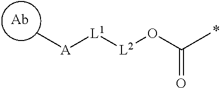

where the asterisk indicates the point of attachment to the PBD, Ab is the antibody, L¹ is a cleavable linker, A is a connecting group connecting L¹ to the antibody, L² is a covalent bond or together with —OC(═O)— forms a self-immolative linker.

In some of these embodiments, L¹ is enzyme cleavable.

It has previously been shown that such ADCs are useful in the treatment of CD25 expressing cancers (see, for example, WO2014/057119, which is incorporated by reference herein in its entirety).

The term anti-CD25-ADC may include any embodiment described in WO 2014/057119. In particular, in preferred embodiments the ADC may have the chemical structure:

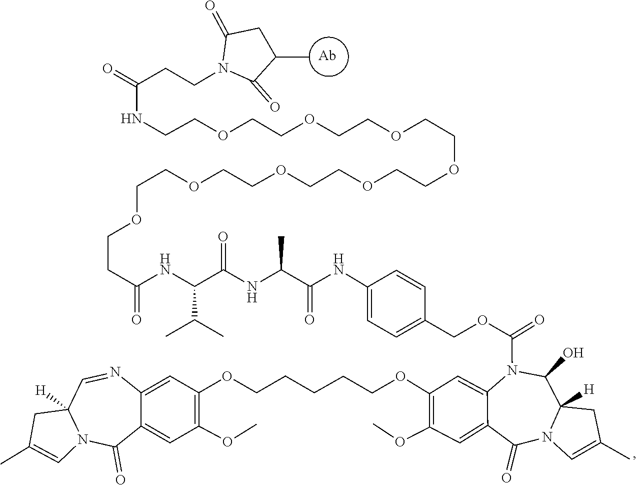

where the Ab is a CD25 antibody, and the DAR is between 1 and 8.

The antibody may comprise a VH domain comprising a VH CDR1 with the amino acid sequence of SEQ ID NO.3, a VH CDR2 with the amino acid sequence of SEQ ID NO.4, and a VH CDR3 with the amino acid sequence of SEQ ID NO.5.

In some aspects the antibody component of the anti-CD25-ADC is an antibody comprising: a VH domain comprising a VH CDR1 with the amino acid sequence of SEQ ID NO.3, a VH CDR2 with the amino acid sequence of SEQ ID NO.4, and a VH CDR3 with the amino acid sequence of SEQ ID NO.5. In some embodiments the antibody comprises a VH domain having the sequence according to SEQ ID NO. 1.

The antibody may further comprise: a VL domain comprising a VL CDR1 with the amino acid sequence of SEQ ID NO.6, a VL CDR2 with the amino acid sequence of SEQ ID NO.7, and a VL CDR3 with the amino acid sequence of SEQ ID NO.8. In some embodiments the antibody further comprises a VL domain having the sequence according to SEQ ID NO. 2.

In some embodiments the antibody comprises a VH domain and a VL domain, the VH and VL domains having the sequences of SEQ ID NO. 1 paired with SEQ ID NO. 2.

The VH and VL domain(s) may pair so as to form an antibody antigen binding site that binds CD25.

In preferred embodiments the antibody is an intact antibody comprising a VH domain and a VL domain, the VH and VL domains having sequences of SEQ ID NO. 1 and SEQ ID NO. 2.

In some embodiments the antibody is a fully human monoclonal IgG1 antibody, preferably IgG1,κ.

In some embodiments the antibody is the AB12 antibody described in WO 2004/045512 (Genmab A/S).

In an aspect the antibody is an antibody as described herein which has been modified (or further modified) as described below. In some embodiments the antibody is a humanised, deimmunised or resurfaced version of an antibody disclosed herein.

The preferred anti-CD25-ADC for use with the aspects of the present disclosure is ADCx25, ADCT-301, or Camidanlumab tesirine as described herein below.

ADCx25

ADCx25 is an antibody drug conjugate composed of a human antibody against human CD25 attached to a pyrrolobenzodiazepine (PBD) warhead via a cleavable linker. The mechanism of action of ADCX25 depends on CD25 binding. The CD25 specific antibody targets the antibody drug conjugate (ADC) to cells expressing CD25. Upon binding, the ADC internalizes and is transported to the lysosome, where the protease sensitive linker is cleaved and free PBD dimer is released inside the target cell. The released PBD dimer inhibits transcription in a sequence-selective manner, due either to direct inhibition of RNA polymerase or inhibition of the interaction of associated transcription factors. The PBD dimer produces covalent crosslinks that do not distort the DNA double helix and which are not recognized by nucleotide excision repair factors, allowing for a longer effective period (Hartley 2011).

It has the chemical structure:

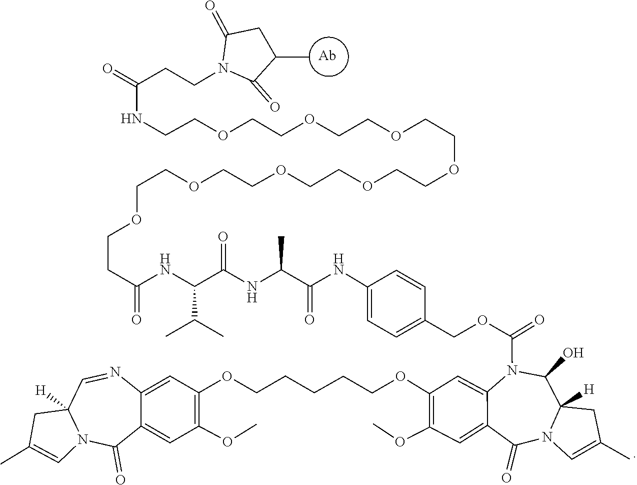

Ab represents Antibody AB12 (fully human monoclonal IgG1, K antibody with the VH and VL sequences SEQ ID NO. 1 and SEQ ID NO. 2, respectively, also known as HuMax-TAC). It is synthesised as described in WO 2014/057119 (Conj AB12-E) and typically has a DAR (Drug to Antibody Ratio) of 2.0+/−0.3.

ADCT-301 or Camidanlumab Tesirine

This ADC is identified by the following designations:
(i) CAS Number→1853239-04-9 (see http://www.cas.org/content/chemical-substances/faqs)
(ii) Unique Ingredient Identifier (UNII)→LYJ1AEJ9YH (see http://www.fda.gov/ForIndustry/DataStandards/SubstanceRegistrationSystem-UniqueIngredientIdentifierUNII/default.htm)

CD25 Binding

The “first target protein” (FTP) as used herein is preferably CD25.

As used herein, “binds CD25” is used to mean the antibody binds CD25 with a higher affinity than a non-specific partner such as Bovine Serum Albumin (BSA, Genbank accession no. CAA76847, version no. CAA76847.1 GI:3336842, record update date: Jan. 7, 2011 02:30 PM). In some embodiments the antibody binds CD25 with an association constant (Kₐ) at least 2, 3, 4, 5, 10, 20, 50, 100, 200, 500, 1000, 2000, 5000, 10⁴, 10⁵ or 10⁶-fold higher than the antibody's association constant for BSA, when measured at physiological conditions. The antibodies of the disclosure can bind CD25 with a high affinity. For example, in some embodiments the antibody can bind CD25 with a K_{D} equal to or less than about 10⁻⁶ M, such as equal to or less than one of 1×10⁻⁶, 10⁻⁷, 10⁻⁸, 10⁻⁹, 10⁻¹⁰, 10⁻¹¹, 10⁻¹², 10⁻¹³ or 10⁻¹⁴.

In some embodiments, CD25 polypeptide corresponds to Genbank accession no. NP_000408, version no. NP_000408.1 GI:4557667, record update date: Sep. 9, 2012 04:59 PM. In one embodiment, the nucleic acid encoding CD25 polypeptide corresponds to Genbank accession no. NM_000417, version no. NM_000417.2 GI:269973860, record update date: Sep. 9, 2012 04:59 PM. In some embodiments, CD25 polypeptide corresponds to Uniprot/Swiss-Prot accession No. P01589.

CARs

As used herein, the term Chimeric antigen receptors (CARs) has its normal meaning in the art, including The current state of the art with respect to CARs is reviewed in for example: Hartmann, J., et al., EMBO Mol. Med., 2017, Vol. 9, Issue 9, pp. 1183-1197//Brudno, J N. & Kochendorfer, J N., 2016, Blood, vol. 127, no. 26, pp. 3321-3330//Zhang and Xu, J. Hematology & Oncology, 2017, 10:1, DOI 10.1186/s13045-016-0379-6//references cited therein.

Briefly, all CARs are composed of an extracellular binding domain, a hinge region, a transmembrane domain, and one or more intracellular signaling domains. Single-chain variable fragments (scFvs) derived from tumor antigen-reactive antibodies are typically used as extracellular binding domains. All CARs harbor the CD3epsilon chain domain as the intracellular signaling domain.

Like antibodies, the extracellular binding domain of a CAR is capable of recognizing and binding to a specific antigen. (Janeway, C., Travers, P., Walport, M., Shlomchik (2001) Immuno Biology, 5th Ed., Garland Publishing, New York). A target antigen generally has numerous binding sites, also called epitopes, recognized by Complementarity Determining Regions (CDRs) on multiple CARs. Each CAR that specifically binds to a different epitope has a different structure. Thus, one antigen may have more than one corresponding CAR. A CAR may comprise a full-length immunoglobulin molecule or an immunologically active portion of a full-length immunoglobulin molecule, i.e., a molecule that contains an antigen binding site that immunospecifically binds an antigen of a target of interest or part thereof (eg. An scFv), such targets including but not limited to, cancer cells.

In addition to the CD3 epsilon chain, second- and third-generation CARs also contain one or more co-stimulatory domains, like CD28 and/or 4-1BB. These co-stimulatory domains improve proliferation, cytokine secretion, resistance to apoptosis, and in vivo persistence of cells expressing the CARs. Third-generation CARs exhibit improved effector functions and in vivo persistence as compared to second-generation CARs.

Fourth-generation CARs, so-called TRUCKs or armored CARs, combine the expression of a second-generation CAR with factors that enhance anti-tumoral activity, such as cytokines, costimulatory ligands, or enzymes that degrade the extracellular matrix of solid tumors (Chmielewski & Abken, ibid.).

Further varieties of CARs exist with enhanced safety features. These include so-called smart T cells which are either equipped with a suicide gene or include synthetic control devices are under non-clinical and clinical investigation (Zhang & Xu, ibid.).

CAR Expressing Immune Cells

Conventional immune cells may be genetically modified such that they express a CAR as described and referenced herein. An immune cells so modified is described herein as “CAR immune cell” and is suitable for use in CAR cell therapies as described in the CAR review references cited above.

CARs may be expressed in a number of different immune cells. Suitable immune cells for expressing a CAR include T-cells—such as cytotoxic T-cells, helper T-cells, and regulatory T-cells—as well as Natural Killer (NK) cells.

In some cases, the CAR immune cell is autologous to the individual treated with the CAR immune cells.

In some cases, the CAR immune cell is allogenic to the individual treated with the CAR immune cells.

CAR T-Cells

In preferred cases, the immune cell is a T-cell. In these cases, the CAR expressing T-cell is termed here in as a “CAR T-cell”.

CAR Immune Cell Toxicity

The term “CAR immune cell toxicity” is used herein to describe any toxicity, pathology, or disorder that can be attributed to the action of CAR immune cells administered to an individual.

Recognised types of CAR immune cell toxicity include:

Cytokine Release Syndrome (CRS)
Individuals with CRS may have one or more of the following symptoms: elevated IL-6, elevated interferon gamma, elevated tumour necrosis factor, pyrexia, fatigue, nausea, tachycardia, hypotension, dyspnea, shortness of breath, pulmonary edema, or cardiac dysfunction.

Neurotoxicity
Individuals with neurotoxicity may have one or more of the following symptoms:
   cerebral oedema, confusion, delirium, aphasia, or encephalopathy.

Tumour Lysis Syndrome (TLS)
Individuals with TLS may have one or more of the following symptoms:
   Hyperuricemia, hyperkalemia, hyperphosphatemia, hypocalcemia, acute uric acid nephropathy, acute kidney failure, seizures, cardiac arrhythmias.

Cellular and/or Humoral Immune Responses
Individuals with an immune response may have one or more of the following symptoms:
   anaphylaxis;

On-Target, Off-Tumour Recognition
The toxic effects of on-target, off-tumour recognition depend upon which normal tissues are subject to the CAR immune cell toxicity.
For example, in individuals treated with CAR immune cells targeting B-cell malignancies (such as anti-CD19 CAR immune cells)), on-target, off-tumour toxicity may result in long term B-cell aplasia due to targeting of healthy B-cells (which express low levels of CD19) by the CAR immune cells.

Off-Target, Off-Tumour Recognition

The toxicity may occur shortly after CAR immune cells are first administered to the individual, for example not more than 1 hour, 2 hours, 4 hours, 8 hours, 24 hours, or 48 hours after CAR immune cells are first administered to the individual.

The toxicity may occur shortly after the second, third, or subsequent dose of CAR immune cells are administered to the individual, for example not more than 1 hour, 2 hours, 4 hours, 8 hours, 24 hours, or 48 hours after the second, third, or subsequent dose of CAR immune cells are administered to the individual.

In some cases the ADC may be administered prophylactically following the successful conclusion of CAR immune cell treatment. That is, the ADC may be administered to the individual before the individual has, or is determined to have, CAR immune cell toxicity. This may be done with a view to, for example, removing the possibility of CAR immune cell toxicity arising in the individual at a future time point due to the presence of persisting CAR immune cells. In some cases CAR immune cell treatment is considered to be successfully concluded when the individual has reached complete response (CR).

Patient Selection

In certain cases, the individuals are selected as suitable for treatment with the ADC before the treatment is administered.

As used herein, individuals who are considered suitable for treatment are those individuals who are expected to benefit from, or respond to, the treatment. For example, individuals who have received at least one dose of CAR immune cells and may have, or be suspected of having, or be at risk of having CAR immune cell toxicity. Individuals may have received a diagnosis of CAR immune cell toxicity. In particular, individuals who may have, or be suspected of having, or be at risk of having, CRS, Neurotoxicity, TLS, or on-target/off-tumour toxicity.

In some cases, individuals are selected on the basis of the amount or pattern of expression of a biomarker in a sample taken from the individual. In some cases, the biomarker is an interleukin, such as IL-6, an interferon, such as interferon gamma, or TNF.

In some cases, expression of the biomarker in a particular tissue of interest is determined. In some cases, systemic expression of the biomarker is determined. For example, in a sample of circulating fluid such as blood, plasma, serum or lymph.

In some aspects, the individual is selected as suitable for treatment due to the presence of biomarker expression in a sample. In those cases, individuals without biomarker expression may be considered not suitable for treatment.

In other aspects, the level of biomarker expression is used to select an individual as suitable for treatment. Where the level of expression of the biomarker is above a threshold level, the individual is determined to be suitable for treatment.

In some aspects, a patient is determined to be suitable for treatment if the level of one or more biomarkers in a sample is at least 2-fold, at least 5-fold, at least 10-fold, at least 20-fold, at least 50-fold, or at least 100-fold the control level.

Samples The sample may comprise or may be derived from: a quantity of blood; a quantity of serum derived from the individual's blood which may comprise the fluid portion of the blood obtained after removal of the fibrin clot and blood cells; a quantity of pancreatic juice; a tissue sample or biopsy; or cells isolated from said individual.

A sample may be taken from any tissue or bodily fluid. In certain aspects, the sample may include or may be derived from a tissue sample, biopsy, resection or isolated cells from said individual.

In certain aspects, the sample is a tissue sample.

In some aspects the sample is taken from a bodily fluid, more preferably one that circulates through the body. Accordingly, the sample may be a blood sample or lymph sample. In some cases, the sample is a urine sample or a saliva sample.

In some cases, the sample is a blood sample or blood-derived sample. The blood derived sample may be a selected fraction of a individual's blood, e.g. a selected cell-containing fraction or a plasma or serum fraction.

A selected cell-containing fraction may contain cell types of interest which may include white blood cells (WBC), particularly peripheral blood mononuclear cells (PBC) and/or granulocytes, and/or red blood cells (RBC).

The sample may be fresh or archival. For example, archival tissue may be from the first diagnosis of an individual, or a biopsy at a relapse. In certain aspects, the sample is a fresh biopsy.

Individual Status

The individual may be an animal, mammal, a placental mammal, a marsupial (e.g., kangaroo, wombat), a monotreme (e.g., duckbilled platypus), a rodent (e.g., a guinea pig, a hamster, a rat, a mouse), murine (e.g., a mouse), a lagomorph (e.g., a rabbit), avian (e.g., a bird), canine (e.g., a dog), feline (e.g., a cat), equine (e.g., a horse), porcine (e.g., a pig), ovine (e.g., a sheep), bovine (e.g., a cow), a primate, simian (e.g., a monkey or ape), a monkey (e.g., marmoset, baboon), an ape (e.g., gorilla, chimpanzee, orangutang, gibbon), or—preferably—a human.

Furthermore, the individual may be any of its forms of development, for example, a foetus. In one preferred embodiment, the individual is a human. The terms “subject”, “patient” and “individual” are used interchangeably herein.

In some cases disclosed herein, an individual has, or is suspected as having, or has been identified as being at risk of, CAR immune cell toxicity. In some cases disclosed herein, the individual has already received a diagnosis of CAR immune cell toxicity.

The individual will typically have received at least one dose of CAR immune cells as treatment for a disorder. That is, a CAR immune cell will typically have been administered to the individual prior to administration of the ADC. In some cases the CAR immune cell has been administered to the individual at least 1 hour, 2 hours, 4 hours, 8 hours, 24 hours, 48 hours, 72 hours, 1 week, 2 weeks, 4 weeks, 3 months, 6 months, 12 months, 2 years, or 5 years prior to administration of the ADC.

In preferred cases the CAR cell expresses, or may express, CD25.

In some cases, the administered CAR cell may specifically bind a tumour associated antigen. In some cases the administered CAR cell may specifically bind CD19, CD20, CD22, BCMA or a solid tumor target.

As used herein, “specifically binds [antigen]” is used to mean the CAR binds [antigen] with a higher affinity than a non-specific partner such as Bovine Serum Albumin (BSA, Genbank accession no. CAA76847, version no. CAA76847.1 GI:3336842, record update date: Jan. 7, 2011 02:30 PM). In some embodiments the antibody binds [antigen] with an association constant (Kₐ) at least 2, 3, 4, 5, 10, 20, 50, 100, 200, 500, 1000, 2000, 5000, 10⁴, 10⁵ or 10⁶-fold higher than the antibody's association constant for BSA, when measured at physiological conditions. CAR cells typically bind [antigen] with a high affinity. For example, in some embodiments the antibody can bind [antigen] with a K_{D} equal to or less than about 10⁻⁶ M, such as equal to or less than one of 1×10⁻⁶, 10⁻⁷, 10⁻⁸, 10⁻⁹, 10⁻¹⁰, 10⁻¹¹, 10⁻¹², 10⁻¹³ or 10⁻¹⁴.

In some embodiments, CD19 polypeptide corresponds to Genbank accession no. NP_001171569, version no. NP_001171569.1 GI:296010921, record update date: Sep. 10, 2012 12:43 AM. In one embodiment, the nucleic acid encoding CD19 polypeptide corresponds to Genbank accession no NM_001178098, version no. NM_001178098.1 GI:296010920, record update date: Sep. 10, 2012 12:43 AM. In some embodiments, CD19 polypeptide corresponds to Uniprot/Swiss-Prot accession No. P15391.

In some embodiments, CD20 polypeptide corresponds to Genbank accession no. CAA31046, version no. CAA31046.1, record update date: Feb. 2, 2011 10:09 AM. In one embodiment, the nucleic acid encoding CD20 polypeptide corresponds to Genbank accession no X12530, version no. X12530.1, record update date: Feb. 2, 2011 10:09 AM. In some embodiments, CD20 polypeptide corresponds to Uniprot/Swiss-Prot accession No. P11836.

In some embodiments, CD22 polypeptide corresponds to Genbank accession no. BAB15489, version no. BAB15489.1 GI:10439338, record update date: Sep. 11, 2006 11:24 PM. In one embodiment, the nucleic acid encoding CD22 polypeptide corresponds to Genbank accession no AK026467, version no. AK026467.1 GI:10439337, record update date: Sep. 11, 2006 11:24 PM.

In some embodiments, solid tumour targets include the specific antigens identified in Yu, S. et al., Journal of Hematology & Oncology 201710:78 (doi.org/10.1186/s13045-017-0444-9). In particular, the specific antigens include those identified in Table 1 of the above references, which is reproduced below:

| Antigen | Full name | Disease |
|---|---|---|
| EGFR | Epidermal growth | NSCLC, epithelial carcinoma, |
| | factor receptor | glioma |
| EGFRvIII | Variant III of the epidermal | Glioblastoma |
| | growth factor receptor | |
| HER2 | Human epidermal growth | Ovarian cancer, breast cancer, |
| | factor receptor 2 | glioblastoma, colon cancer, |
| | | osteosarcoma, medulloblastoma |
| MSLN | Mesothelin | Mesothelioma, ovarian cancer, |
| | | pancreatic adenocarcinoma |
| PSMA | Prostate-specific membrane | Prostate cancer |
| | antigen | |
| CEA | Carcinoembryonic antigen | Pancreatic adenocarcinoma, |
| | | breast cancer, |
| | | colorectal carcinoma |
| GD2 | Disialoganglioside 2 | Neuroblastoma, melanoma |
| IL13Rα2 | Interleukin-13Ra2 | Glioma |
| GPC3 | Glypican-3 | Hepatocellular carcinoma |
| CAIX | Carbonic anhydrase IX | Renal cell carcinoma (ROC) |
| L1-CAM | L1 cell adhesion molecule | Neuroblastoma, melanoma, |
| | | ovarian adenocarcinoma |
| CA125 | Cancer antigen 125 (also | Epithelial ovarian cancers |
| | known as MUC16) | |
| CD133 | Cluster of differentiation 133 | Glioblastoma, |
| | (also known as prominin-1) | cholangiocarcinoma |
| | | (CCA) |
| FAP | Fibroblast activation protein | Malignant pleural |
| | | mesothelioma (MPM) |
| CTAG1B | Cancer/testis antigen 1B (also | Melanoma and ovarian cancer |
| | known as NY-ESO-1) | |
| MUC1 | Mucin 1 | Seminal vesicle cancer |
| FR-α | Folate receptor-α | Ovarian cancer |

In some cases, the disorder treated by the CAR immune cell may be, or have been, any disorder treatable with CAR immune cells, for example, a proliferative disorder such as cancer or haematological cancer. The treated disorder may be, or have been, (classical) Hodgkins lymphoma (mixed cellularity type), or non-Hodgkins lymphoma (including B-cell chronic lymphatic leukemia, diffuse large B-cell lymphoma (DLBCL), follicular lymphoma, (FL), Mantle Cell lymphoma (MCL), chronic lymphatic lymphoma (CLL), Marginal Zone B-cell lymphoma (MZBL) and leukemias such as Hairy cell leukemia (HCL), Hairy cell leukemia variant (HCL-v), Acute Myeloid Leukaemia (AML), Acute Lymphoblastic Leukaemia (ALL) such as Philadelphia chromosome-positive ALL (Ph+ALL) or Philadelphia chromosome-negative ALL (Ph−ALL) [Fielding A., Haematologica. 2010 January; 95(1): 8-12], small cell lymphocytic lymphoma, adult T-cell leukemia/lymphoma, and anaplastic large cell lymphoma.

The individual may have reached complete response (CR) to the disorder or may have been determined to have reached complete response (CR) to the disorder.

‘Complete response’ is used herein to mean the absence of any clinical evidence of disease in an individual. Evidence may be assessed using the appropriate methodology in the art, for example CT or PET scanning, or biopsy where appropriate.

Controls

In some aspects, biomarker expression in the individual is compared to biomarker expression in a control. Controls are useful to support the validity of staining, and to identify experimental artefacts.

In some cases, the control may be a reference sample or reference dataset. The reference may be a sample that has been previously obtained from a individual with a known degree of suitability. The reference may be a dataset obtained from analyzing a reference sample.

Controls may be positive controls in which the biomarker is known to be present, or expressed at high level, or negative controls in which the biomarker is known to be absent or expressed at low level.

In some cases the control may be a sample obtained from the same individual as the test sample, but from before they had, or were suspected to have, CAR immune cell toxicity. For example, the control may be a sample obtained from the same individual as the test sample, but from before they were first administered CAR immune cells.

In some cases, the control is standard reference dataset for normal biomarker levels.

In some cases, a test sample is analyzed prior to incubation with an antibody to determine the level of background staining inherent to that sample.

In some cases an isotype control is used. Isotype controls use an antibody of the same class as the target specific antibody, but are not immunoreactive with the sample. Such controls are useful for distinguishing non-specific interactions of the target specific antibody.

The methods may include hematopathologist interpretation of morphology and immunohistochemistry, to ensure accurate interpretation of test results. The method may involve confirmation that the pattern of expression correlates with the expected pattern. For example, where the amount of a first target protein and/or a second target protein expression is analyzed, the method may involve confirmation that in the test sample the expression is observed as membrane staining, with a cytoplasmic component. The method may involve confirmation that the ratio of target signal to noise is above a threshold level, thereby allowing clear discrimination between specific and non-specific background signals.

Methods of Treatment

The term “treatment,” as used herein in the context of treating a condition, pertains generally to treatment and therapy, whether of a human or an animal (e.g., in veterinary applications), in which some desired therapeutic effect is achieved, for example, the inhibition of the progress of the condition, and includes a reduction in the rate of progress, a halt in the rate of progress, regression of the condition, amelioration of the condition, and cure of the condition. Treatment as a prophylactic measure (i.e., prophylaxis, prevention) is also included.

The term “therapeutically-effective amount” or “effective amount” as used herein, pertains to that amount of an active compound, or a material, composition or dosage from comprising an active compound, which is effective for producing some desired therapeutic effect, commensurate with a reasonable benefit/risk ratio, when administered in accordance with a desired treatment regimen.

Similarly, the term “prophylactically-effective amount,” as used herein, pertains to that amount of an active compound, or a material, composition or dosage from comprising an active compound, which is effective for producing some desired prophylactic effect, commensurate with a reasonable benefit/risk ratio, when administered in accordance with a desired treatment regimen.

Disclosed herein are methods of therapy. Also provided is a method of treatment, comprising administering to a subject in need of treatment a therapeutically-effective amount of an ADC. The term “therapeutically effective amount” is an amount sufficient to show benefit to a subject. Such benefit may be at least amelioration of at least one symptom. The actual amount administered, and rate and time-course of administration, will depend on the nature and severity of what is being treated. Prescription of treatment, e.g. decisions on dosage, is within the responsibility of general practitioners and other medical doctors. The subject may have been tested to determine their eligibility to receive the treatment according to the methods disclosed herein. The method of treatment may comprise a step of determining whether a subject is eligible for treatment, using a method disclosed herein.

The ADC may comprise an anti-CD25 antibody. The anti-CD25 antibody may be HuMax-TAC™. The ADC may comprise a drug which is a PBD dimer. The ADC may be a anti-CD25-ADC, and in particular, ADCX25, ADCT 301, or Camidanlumab tesirine. The ADC may be an ADC disclosed in WO2014/057119.

The treatment may involve administration of the ADC alone or in further combination with other treatments, either simultaneously or sequentially dependent upon the condition to be treated. Examples of treatments and therapies include, but are not limited to, chemotherapy (the administration of active agents, including, e.g. drugs, such as therapeutic agents).

A “therapeutic agent” is a chemical compound useful in the treatment of CAR immune cell toxicity, regardless of mechanism of action. Classes of therapeutic agents include, but are not limited to: antibodies, immune regulators, and immune suppressors.

An example category of therapeutic agents useful in the present disclosure are the steroids. Preferably, the steroid is dexamethasone. Other suitable steroid are found in the classes of corticosteroids, such as glucocorticoids. Example glucocorticoids are Cortisol (hydrocortisone), Cortisone, Prednisone, Prednisolone, Methylprednisolone, Dexamethasone, Betamethasone, Triamcinolone, Fludrocortisone acetate, and Deoxycorticosterone acetate.

Dexamethasone:
(iii) CAS Number→50-02-2 (see http://www.cas.org/content/chemical-substances/faqs)
(iv) Unique Ingredient Identifier (UNII)→7S5I7G3JQL (see http://www.fda.gov/ForIndustry/DataStandards/SubstanceRedistrationSystem-UniqueIngredientIdentifierUNII/default.htm)
(v) IUPAC name→(8S,9R,10S,11S,13S,14S,16R,17R)-9-Fluoro-11,17-dihydroxy-17-(2-hydroxyacetyl)-10,13,16-trimethyl-6,7,8,9,10,11,12,13,14,15,16,17-dodecahydro-3H-cyclopenta[a]phenanthren-3-one
(vi) Structure 4

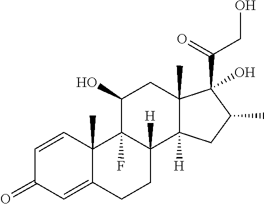

Another example category of therapeutic agent are agents which inhibit antagonize immunostimulatory molecules such as interleukins and interferons. An example of such an agent is Tocilizumab, an antibody which antagonized IL-6 signalling through binding to the IL-6 receptor.

Tocilizumab:
(i) CAS Number→375823-41-9 (see http://www.cas.org/content/chemical-substances/faqs)
(ii) Unique Ingredient Identifier (UNII)→I031V2H011 (see http://www.fda.gov/ForIndustry/DataStandards/SubstanceRegistrationSystem-UniqueIngredientIdentifierUNII/default.htm)

Compositions according to the present disclosure are preferably pharmaceutical compositions. Pharmaceutical compositions according to the present disclosure, and for use in accordance with the present disclosure, may comprise, in addition to the active ingredient, i.e. a conjugate compound, a pharmaceutically acceptable excipient, carrier, buffer, stabiliser or other materials well known to those skilled in the art. Such materials should be non-toxic and should not interfere with the efficacy of the active ingredient. The precise nature of the carrier or other material will depend on the route of administration, which may be oral, or by injection, e.g. cutaneous, subcutaneous, or intravenous.

Pharmaceutical compositions for oral administration may be in tablet, capsule, powder or liquid form. A tablet may comprise a solid carrier or an adjuvant. Liquid pharmaceutical compositions generally comprise a liquid carrier such as water, petroleum, animal or vegetable oils, mineral oil or synthetic oil. Physiological saline solution, dextrose or other saccharide solution or glycols such as ethylene glycol, propylene glycol or polyethylene glycol may be included. A capsule may comprise a solid carrier such a gelatin.

For intravenous, cutaneous or subcutaneous injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilisers, buffers, antioxidants and/or other additives may be included, as required.

Dosage

Ideally, the ADC is administered in a dosage regime sufficient to reduce the number and/or activity of activated CAR immune cells, thereby alleviating CAR immune cell toxicity. In some cases the administered dose(s) of ADC is sufficient to effectively eliminate activated CAR immune cells from the individual. In some cases the administered dose(s) of ADC is sufficient to effectively eliminate all CAR immune cells (including inactive CAR cells) from the individual. The elimination of CAR T-cells may be assesses by the absence of detectable CAR T-cells in a sample taken from the individual.

It will be appreciated by one of skill in the art that appropriate dosages of the ADC and/or the secondary agent, and compositions comprising these active elements, can vary from subject to subject. Determining the optimal dosage will generally involve the balancing of the level of therapeutic benefit against any risk or deleterious side effects. The selected dosage level will depend on a variety of factors including, but not limited to, the activity of the particular compound, the route of administration, the time of administration, the rate of excretion of the compound, the duration of the treatment, other drugs, compounds, and/or materials used in combination, the severity of the condition, and the species, sex, age, weight, condition, general health, and prior medical history of the subject. The amount of compound and route of administration will ultimately be at the discretion of the physician, veterinarian, or clinician, although generally the dosage will be selected to achieve local concentrations at the site of action which achieve the desired effect without causing substantial harmful or deleterious side-effects.

In certain aspects, the dosage of ADC is determined by the expression of a first target protein observed in a sample obtained from the subject. Thus, the level or localisation of expression of the first target protein in the sample may be indicative that a higher or lower dose of ADC is required. For example, a high expression level of the first target protein may indicate that a higher dose of ADC would be suitable. In some cases, a high expression level of the first target protein may indicate the need for administration of another agent in addition to the ADC. For example, administration of the ADC in conjunction with a therapeutic agent. A high expression level of the first target protein may indicate a more aggressive therapy.

Administration can be effected in one dose, continuously or intermittently (e.g., in divided doses at appropriate intervals) throughout the course of treatment. Methods of determining the most effective means and dosage of administration are well known to those of skill in the art and will vary with the formulation used for therapy, the purpose of the therapy, the target cell(s) being treated, and the subject being treated. Single or multiple administrations can be carried out with the dose level and pattern being selected by the treating physician, veterinarian, or clinician.

In some cases the ADC is given as a single dose. In some cases it is given as a double dose separated by, for example, 24 hours, 48 hours, 73 hours, 1 week, 2 weeks, or 3 weeks. In some cases, each dose is of equal size.

Each dose may be about 10, 20, 30, 40, 50, 60, 70, 80, 90, 100, 120, 150, 200, 250, or 300 μg/kg. In some cases the total dose is 1 to 10 μg/kg, 11 to 20 μg/kg, 21 to 30 μg/kg, 31 to 40 μg/kg, 41 to 50 μg/kg, 51 to 60 μg/kg, 61 to 70 μg/kg, 71 to 80 μg/kg, 81 to 90 μg/kg, 91 to 100 μg/kg, 101 to 120 μg/kg, 121 to 140 μg/kg, 141 to 160 μg/kg, 161 to 180 μg/kg, 181 to 200 μg/kg, 201 to 220 μg/kg, 221 to 240 μg/kg, 241 to 260 μg/kg, 261 to 280 μg/kg, or 281 to 300 μg/kg.

The first target protein is preferably CD25. The ADC may comprise an anti-CD25 antibody. The anti-CD25 antibody may be HuMax-TAC™. The ADC may comprise a drug which is a PBD dimer. The ADC may be an anti-CD25-ADC, and in particular, is preferably ADCX25, ADCT-301, or Camidanlumab tesirine. The ADC may be an ADC disclosed in WO2014/057119.

Antibodies

The term “antibody” herein is used in the broadest sense and specifically covers monoclonal antibodies, polyclonal antibodies, dimers, multimers, multispecific antibodies (e.g., bispecific antibodies), intact antibodies (also described as “full-length” antibodies) and antibody fragments, so long as they exhibit the desired biological activity, for example, the ability to bind a first target protein (Miller et al (2003) Jour. of Immunology 170:4854-4861). Antibodies may be murine, human, humanized, chimeric, or derived from other species such as rabbit, goat, sheep, horse or camel.

An antibody is a protein generated by the immune system that is capable of recognizing and binding to a specific antigen. (Janeway, C., Travers, P., Walport, M., Shlomchik (2001) Immuno Biology, 5th Ed., Garland Publishing, New York). A target antigen generally has numerous binding sites, also called epitopes, recognized by Complementarity Determining Regions (CDRs) on multiple antibodies. Each antibody that specifically binds to a different epitope has a different structure. Thus, one antigen may have more than one corresponding antibody. An antibody may comprise a full-length immunoglobulin molecule or an immunologically active portion of a full-length immunoglobulin molecule, i.e., a molecule that contains an antigen binding site that immunospecifically binds an antigen of a target of interest or part thereof, such targets including but not limited to, cancer cell or cells that produce autoimmune antibodies associated with an autoimmune disease. The immunoglobulin can be of any type (e.g. IgG, IgE, IgM, IgD, and IgA), class (e.g. IgG1, IgG2, IgG3, IgG4, IgA1 and IgA2) or subclass, or allotype (e.g. human G1m1, G1m2, G1m3, non-G1m1 [that, is any allotype other than G1m1], G1m17, G2m23, G3m21, G3m28, G3m11, G3m5, G3m13, G3m14, G3m10, G3m15, G3m16, G3m6, G3m24, G3m26, G3m27, A2m1, A2m2, Km1, Km2 and Km3) of immunoglobulin molecule. The immunoglobulins can be derived from any species, including human, murine, or rabbit origin.

“Antibody fragments” comprise a portion of a full length antibody, generally the antigen binding or variable region thereof. Examples of antibody fragments include Fab, Fab′, F(ab′)₂, and scFv fragments; diabodies; linear antibodies; fragments produced by a Fab expression library, anti-idiotypic (anti-Id) antibodies, CDR (complementary determining region), and epitope-binding fragments of any of the above which immunospecifically bind to cancer cell antigens, viral antigens or microbial antigens, single-chain antibody molecules; and multispecific antibodies formed from antibody fragments.

The term “monoclonal antibody” as used herein refers to an antibody obtained from a population of substantially homogeneous antibodies, i.e. the individual antibodies comprising the population are identical except for possible naturally occurring mutations that may be present in minor amounts. Monoclonal antibodies are highly specific, being directed against a single antigenic site. Furthermore, in contrast to polyclonal antibody preparations which include different antibodies directed against different determinants (epitopes), each monoclonal antibody is directed against a single determinant on the antigen. In addition to their specificity, the monoclonal antibodies are advantageous in that they may be synthesized uncontaminated by other antibodies. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present disclosure may be made by the hybridoma method first described by Kohler et al (1975) Nature 256:495, or may be made by recombinant DNA methods (see, U.S. Pat. No. 4,816,567). The monoclonal antibodies may also be isolated from phage antibody libraries using the techniques described in Clackson et al (1991) Nature, 352:624-628; Marks et al (1991) J. Mol. Biol., 222:581-597 or from transgenic mice carrying a fully human immunoglobulin system (Lonberg (2008) Curr. Opinion 20(4):450-459).

The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al (1984) Proc. Natl. Acad. Sci. USA, 81:6851-6855). Chimeric antibodies include “primatized” antibodies comprising variable domain antigen-binding sequences derived from a non-human primate (e.g. Old World Monkey or Ape) and human constant region sequences.

An “intact antibody” herein is one comprising VL and VH domains, as well as a light chain constant domain (CL) and heavy chain constant domains, CH1, CH2 and CH3. The constant domains may be native sequence constant domains (e.g. human native sequence constant domains) or amino acid sequence variant thereof. The intact antibody may have one or more “effector functions” which refer to those biological activities attributable to the Fc region (a native sequence Fc region or amino acid sequence variant Fc region) of an antibody. Examples of antibody effector functions include C1q binding; complement dependent cytotoxicity; Fc receptor binding; antibody-dependent cell-mediated cytotoxicity (ADCC); phagocytosis; and down regulation of cell surface receptors such as B cell receptor and BCR.

Depending on the amino acid sequence of the constant domain of their heavy chains, intact antibodies can be assigned to different “classes.” There are five major classes of intact antibodies: IgA, IgD, IgE, IgG, and IgM, and several of these may be further divided into “subclasses” (isotypes), e.g., IgG1, IgG2, IgG3, IgG4, IgA, and IgA2. The heavy-chain constant domains that correspond to the different classes of antibodies are called α, δ, ε, γ, and μ, respectively. The subunit structures and three-dimensional configurations of different classes of immunoglobulins are well known.

### BRIEF DESCRIPTION OF THE FIGURES

Embodiments and experiments illustrating the principles of the disclosure will now be discussed with reference to the accompanying FIGURES in which:

FIG. 1. Sequences

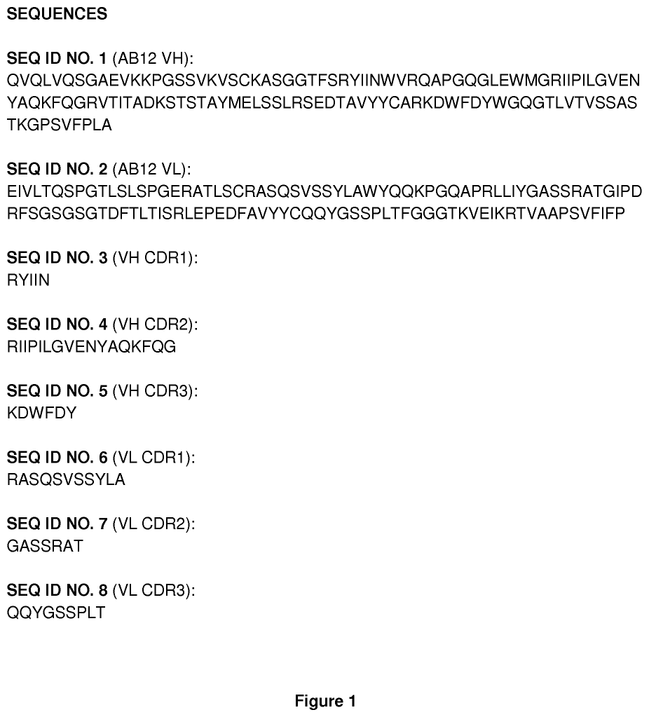

[symbol]

The disclosure includes the combination of the aspects and preferred features described except where such a combination is clearly impermissible or expressly avoided.

The section headings used herein are for organizational purposes only and are not to be construed as limiting the subject matter described.

Aspects and embodiments of the present disclosure will now be illustrated, by way of example, with reference to the accompanying FIGURES. Further aspects and embodiments will be apparent to those skilled in the art. All documents mentioned in this text are incorporated herein by reference.

Throughout this specification, including the claims which follow, unless the context requires otherwise, the word “comprise,” and variations such as “comprises” and “comprising,” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment.

### SOME EMBODIMENTS

1. A method for treating or preventing CAR T-cell toxicity in an individual, the method comprising administering to the individual an effective amount of ADCx25, ADCT 301, or Camidanlumab tesirine.

2. An ADC for use in a method of treatment or prevention of CAR T-cell toxicity in an individual, wherein the treatment comprises administration of the ADCx25, ADCT 301, or Camidanlumab tesirine to the individual.

3. A pharmaceutical composition comprising ADCx25, ADCT 301, or Camidanlumab tesirine for use in a method of treating or preventing CAR T-cell toxicity in an individual, wherein the treatment comprises administration of ADCx25, ADCT 301, or Camidanlumab tesirine to the individual; optionally wherein the composition comprises a pharmaceutically acceptable excipient

4. Use of ADCx25, ADCT 301, or Camidanlumab tesirine in the manufacture of a medicament for treating or preventing CAR T-cell toxicity in an individual, wherein the medicament comprises ADCx25, ADCT 301, or Camidanlumab tesirine, and wherein the treatment comprises administration of the medicament to the individual.

5. A kit comprising:
a first medicament comprising ADCx25, ADCT 301, or Camidanlumab tesirine;
a package insert comprising instructions for administration of the first medicament to an individual for the treatment or prevention of CAR T-cell toxicity.

6. The method, ADC, use, or kit according to any previous paragraph, wherein a CAR T-cell has been administered to the individual prior to administration of the ADC.

7. The method, ADC, use, or kit according to any preceding paragraph, wherein the individual has CAR T-cell toxicity or has been determined to have CAR T-cell toxicity.

8. The method, ADC, use, or kit according to any preceding paragraph, wherein the individual had cancer, or had been determined to have cancer, wherein the individual has reached complete response (CR) or has been determined to have reached complete response (CR).

9. The method, ADC, use, or kit according to any preceding paragraph, wherein the ADC is administered as a single dose, or two separate doses.

10. The method, ADC, use, or kit according to paragraph 17, wherein the second dose is administered 1 week or 3 weeks after the first dose.

11. The method, ADC, use, or kit according to any preceding paragraph, wherein each dose is about 1 to 10 μg/kg, 11 to 20 μg/kg, 21 to 30 μg/kg, 31 to 40 μg/kg, 41 to 50 μg/kg, 51 to 60 μg/kg, 61 to 70 μg/kg, 71 to 80 μg/kg, 81 to 90 μg/kg, 91 to 100 μg/kg, 101 to 120 μg/kg, 121 to 140 μg/kg, 141 to 160 μg/kg, 161 to 180 μg/kg, 181 to 200 μg/kg, 201 to 220 μg/kg, 221 to 240 μg/kg, 241 to 260 μg/kg, 261 to 280 μg/kg, or 281 to 300 μg/kg.

12. The method, ADC, use, or kit according to any previous paragraph, wherein the individual is human.

13. The method, ADC, use, or kit according to any previous paragraph, wherein the CAR immune cell toxicity is one or more disorder selected from the following:
(a) Cytokine Release syndrome (CRS), such as elevated IL-6, elevated interferon gamma, elevated tumour necrosis factor, pyrexia, fatigue, nausea, tachycardia, hypotension, dyspnea, shortness of breath, pulmonary edema, or cardiac dysfunction;
(b) Neurotoxicity, such as cerebral oedema, confusion, delirium, aphasia, or encephalopathy;
(c) Tumour Lysis Syndrome (TLS), such as hyperuricemia, or hyperkalemia;
(d) Cellular and/or humoral immune responses, such as anaphylaxis;
(e) On-target, off-tumour recognition; or
(f) Off-target, off-tumour recognition.

14. The method, ADC, use, or kit according to any previous paragraph wherein the administered CAR immune cell expresses a CAR that specifically binds CD19, CD20, or CD22.

15. A method, ADC, use, or kit according to any previous paragraph, wherein the ADC is administered in combination with a corticosteroid such as dexamethasone, and/or an IL-6 antagonist such as tocilizumab.

### STATEMENTS OF INVENTION

1. A method for treating or preventing CAR immune cell toxicity in an individual, the method comprising administering to the individual an effective amount of an ADC.

2. An ADC for use in a method of treatment or prevention of CAR immune cell toxicity in an individual, wherein the treatment comprises administration of the ADC to the individual.

3. A pharmaceutical composition comprising an ADC for use in a method of treating or preventing CAR immune cell toxicity in an individual, wherein the treatment comprises administration of the ADC to the individual; optionally wherein the composition comprises a pharmaceutically acceptable excipient

4. Use of an ADC in the manufacture of a medicament for treating or preventing CAR immune cell toxicity in an individual, wherein the medicament comprises an ADC, and wherein the treatment comprises administration of the medicament to the individual.

5. A kit comprising:
a first medicament comprising an ADC;
a package insert comprising instructions for administration of the first medicament to an individual for the treatment or prevention of CAR immune cell toxicity.

6. The method, ADC, use, or kit according to any previous paragraph, wherein a CAR immune cell has been administered to the individual prior to administration of the ADC.

7. The method, ADC, use, or kit according to paragraph 6, wherein a CAR immune cell has been administered to the individual at least 1 hour, 2 hours, 4 hours, 8 hours, 24 hours, 48 hours, 72 hours, 1 week, 2 weeks, 4 weeks, 3 months, 6 months, 12 months, 2 years, or 5 years prior to administration of the ADC.

8. The method, ADC, use, or kit according to any preceding paragraph, wherein the individual has CAR immune cell toxicity or has been determined to have CAR immune cell toxicity.

9. The method, ADC, use, or kit according to any one of paragraphs 1 to 8, wherein the individual does not have CAR immune cell toxicity or has not been determined to have CAR immune cell toxicity.

10. The method, ADC, use, or kit according to any preceding paragraph, wherein the individual had a disorder, or had been determined to have a disorder, wherein the individual has reached complete response (CR) to the disorder or has been determined to have reached complete response (CR) to the disorder.

11. The method, ADC, use, or kit according to paragraph 10, wherein the disorder is a proliferative disease.

12. The method, ADC, use, or kit of paragraph 10, wherein the disorder is cancer.

13. The method, ADC, use, or kit of paragraph 10, wherein the cancer is a haematological cancer.

14. The method, ADC, use, or kit of paragraph 10, wherein the disorder is selected from the group comprising: Hodgkin's and non-Hodgkin's Lymphoma, including diffuse large B-cell lymphoma (DLBCL), follicular lymphoma, (FL), Mantle Cell lymphoma (MCL), chronic lymphatic lymphoma (CLL), Marginal Zone B-cell lymphoma (MZBL) and leukemias such as Hairy cell leukemia (HCL), Hairy cell leukemia variant (HCL-v), Acute Myeloid Leukaemia (AML), and Acute Lymphoblastic Leukaemia (ALL) such as Philadelphia chromosome-positive ALL (Ph+ALL) or Philadelphia chromosome-negative ALL (Ph−ALL).

15. The method, ADC, use, or kit according to any preceding paragraph, wherein the ADC is administered in a dosage regime sufficient to eliminate or inactivate CAR immune cells in the individual.

16. The method, ADC, use, or kit according to any preceding paragraph, wherein the ADC is administered as a single dose.

17. The method, ADC, use, or kit according to any one of paragraphs 1 to 15, wherein the ADC is administered as two doses.

18. The method, ADC, use, or kit according to paragraph 17, wherein the second dose is administered 1 week after the first dose.

19. The method, ADC, use, or kit according to paragraph 17, wherein the second dose is administered 3 weeks after the first dose.

20. The method, ADC, use, or kit according to any preceding paragraph, wherein each dose is about 10, 20, 30, 40, 50, 60, 70, 80, 90, 100, 120, 150, 200, 250, or 300 μg/kg.

21. The method, ADC, use, or kit according to any preceding paragraph, wherein each dose is about 1 to 10 μg/kg, 11 to 20 μg/kg, 21 to 30 μg/kg, 31 to 40 μg/kg, 41 to 50 μg/kg, 51 to 60 μg/kg, 61 to 70 μg/kg, 71 to 80 μg/kg, 81 to 90 μg/kg, 91 to 100 μg/kg, 101 to 120 μg/kg, 121 to 140 μg/kg, 141 to 160 μg/kg, 161 to 180 μg/kg, 181 to 200 μg/kg, 201 to 220 μg/kg, 221 to 240 μg/kg, 241 to 260 μg/kg, 261 to 280 μg/kg, or 281 to 300 μg/kg.

22. The method, ADC, use, or kit according to any previous paragraph, wherein the individual is human.

23. The method, ADC, use, or kit according to any previous paragraph, wherein the CAR immune cell toxicity is one or more disorder selected from the following:
(g) Cytokine Release syndrome (CRS), such as elevated IL-6, elevated interferon gamma, elevated tumour necrosis factor, pyrexia, fatigue, nausea, tachycardia, hypotension, dyspnea, shortness of breath, pulmonary edema, or cardiac dysfunction;
(h) Neurotoxicity, such as cerebral oedema, confusion, delirium, aphasia, or encephalopathy;
(i) Tumour Lysis Syndrome (TLS), such as hyperuricemia, or hyperkalemia;
(j) Cellular and/or humoral immune responses, such as anaphylaxis;
(k) On-target, off-tumour recognition; or
(l) Off-target, off-tumour recognition.

24. The method, ADC, use, or kit according to any previous paragraph wherein the administered CAR immune cell expresses a CAR that specifically binds a tumour associated antigen.

25. The method, ADC, use, or kit according to paragraph 24, wherein the administered CAR immune cell expresses a CAR that specifically binds CD19, CD20, or CD22.

26. The method, ADC, use, or kit according to any previous paragraph, wherein the CAR immune cell is a CAR T-cell.

27. The method, ADC, use, or kit according to paragraph 26, wherein the CAR T-cell is a 1^{st} generation CAR T-cell, a 2^{nd} generation CAR T-cell, a 3^{rd} generation CAR T-cell, a 4^{th} generation CAR T-cell, a TRUCK, a smart CAR, or an iCAR.

28. A method, ADC, use, or kit according to any previous paragraph, wherein the ADC is an anti-CD25 ADC.

29. A method, ADC, use, or kit according to paragraph 28, wherein the anti-CD25 ADC is ADCx25, ADCT 301, or Camidanlumab tesirine.

30. A method, ADC, use, or kit according to any previous paragraph, wherein the ADC is administered in combination with a therapeutic agent.

31. A method, ADC, use, or kit according to paragraph 30, wherein the therapeutic agent is a corticosteroid such as dexamethasone, or an IL-6 antagonist such as tocilizumab.

### EXAMPLES

### Example 1: In Vitro Demonstration of CAR T-Cell Killing on Treatment with an Anti-CD25 ADC

CAR-T cells are placed in culture with appropriate aliquots [suggested range of 1 μg/ml to 1 pg/ml] of CD25-ADC to confirm binding of the ADC to CD25 on the CAR-T cell surface, and to determine that killing of the CAR-T cells occurs upon release of the PBD moiety from the ADC.

### Example 2: In Vivo Treatment of Acute CAR-T Toxicity

A patient treated with CD19 CAR-T exhibits acute toxicity within few hours of administration of CAR-T: shortness of breath, confusion, hypotension. The patient is transferred to ICU for general management of consequences of “cytokine storm” and specifically for treatment with tocilizumab.

The patient's condition does not improve despite receiving tocilizumab and intensive care. Administration of CD25-ADC [suggested range 10-50 μg/kg] leads to significant improvement of patient's status, and to eventual full recovery from acute toxic effects of CAR-T administration. Complete elimination of CAR-T cells is confirmed by FACS analysis of patient's blood.

### Example 3: In Vivo Treatment of Chronic CAR-T Toxicity

A patient treated with CD19 CAR-T is considered to be good responder to CAR-T treatment.

Several weeks after cessation of CAR-T dosing, the patient experiences symptoms consistent with chronic depletion of CD-19 positive B-cells. FACS analysis reveals significant clonal expansion of CD19/CD25 positive T-cells. It is determined that depletion of B-cells in this patient is most likely caused by clonal expansion of CD25 positive CAR-T cells.

Administration of CD25-ADC [suggested range 10-50 μg/kg] leads to significant improvement of patient's status, and to eventual full recovery from chronic toxic effects of CAR-T administration. Complete elimination of CAR-T cells is confirmed by FACS analysis of patient's blood.

1.-28. (canceled)

29. A method for treating or preventing CAR immune cell toxicity in an individual, the method comprising administering to the individual an effective amount of an anti-CD25 antibody drug conjugate (ADC).

30. The method according to claim 29, wherein a CAR immune cell has been administered to the individual prior to administration of the ADC.

31. The method according to claim 29, wherein a CAR immune cell has been administered to the individual at least 1 hour, 2 hours, 4 hours, 8 hours, 24 hours, 48 hours, 72 hours, 1 week, 2 weeks, 4 weeks, 3 months, 6 months, 12 months, 2 years, or 5 years prior to administration of the ADC.

32. The method according to claim 29, wherein the individual has CAR immune cell toxicity or has been determined to have CAR immune cell toxicity.

33. The method according to claim 29, wherein the individual does not have CAR immune cell toxicity or has not been determined to have CAR immune cell toxicity.

34. The method according to claim 29, wherein the individual had a disorder, or had been determined to have a disorder, wherein the individual has reached complete response (CR) to the disorder or has been determined to have reached complete response (CR) to the disorder.

35. The method according to claim 34, wherein the disorder is selected from the group consisting of:
(i) a proliferative disease;
(ii) cancer;
(iii) a haematological cancer; and
(iv) Hodgkin's and non-Hodgkin's Lymphoma, including diffuse large B-cell lymphoma (DLBCL), follicular lymphoma, (FL), Mantle Cell lymphoma (MCL), chronic lymphatic lymphoma (CLL), Marginal Zone B-cell lymphoma (MZBL) and leukemias such as Hairy cell leukemia (HCL), Hairy cell leukemia variant (HCL-v), Acute Myeloid Leukaemia (AML), and Acute Lymphoblastic Leukaemia (ALL) such as Philadelphia chromosome-positive ALL (Ph+ALL) or Philadelphia chromosome-negative ALL (Ph−ALL).

36. The method according to claim 29, wherein the anti-CD25 ADC is administered in a dosage regime sufficient to eliminate or inactivate CAR immune cells in the individual.

37. The method according to claim 29, wherein the anti-CD25 ADC is administered as a single dose.

38. The method according to claim 29, wherein the anti-CD25 ADC is administered as two doses.

39. The method according to claim 38, wherein the second dose is administered 1 week or 3 weeks after the first dose.

40. The method according to claim 29, wherein each dose of anti-CD25 ADC is about 10, 20, 30, 40, 50, 60, 70, 80, 90, 100, 120, 150, 200, 250, or 300 μg/kg.

41. The method according to claim 29, wherein each dose of anti-CD25 ADC is about 1 to 10 μg/kg, 11 to 20 μg/kg, 21 to 30 μg/kg, 31 to 40 μg/kg, 41 to 50 μg/kg, 51 to 60 μg/kg, 61 to 70 μg/kg, 71 to 80 μg/kg, 81 to 90 μg/kg, 91 to 100 μg/kg, 101 to 120 μg/kg, 121 to 140 μg/kg, 141 to 160 μg/kg, 161 to 180 μg/kg, 181 to 200 μg/kg, 201 to 220 μg/kg, 221 to 240 μg/kg, 241 to 260 μg/kg, 261 to 280 μg/kg, or 281 to 300 μg/kg.

42. The method according to claim 29, wherein the individual is human.

43. The method according to claim 29, wherein the CAR immune cell toxicity is one or more disorder selected from the following:
(a) Cytokine Release syndrome (CRS), such as elevated IL-6, elevated interferon gamma, elevated tumour necrosis factor, pyrexia, fatigue, nausea, tachycardia, hypotension, dyspnea, shortness of breath, pulmonary edema, or cardiac dysfunction;
bh) Neurotoxicity, such as cerebral oedema, confusion, delirium, aphasia, or encephalopathy;
(c) Tumour Lysis Syndrome (TLS), such as hyperuricemia, or hyperkalemia;
(d) Cellular and/or humoral immune responses, such as anaphylaxis;
(e) On-target, off-tumour recognition; or
(f) Off-target, off-tumour recognition.

44. The method according to claim 30, wherein the administered CAR immune cell expresses a CAR that specifically binds a tumour associated antigen, and/or the administered CAR immune cell expresses a CAR that specifically binds CD19, CD20, or CD22.

45. The method according to claim 29, wherein the CAR immune cell is a CAR T-cell.

46. The method according to claim 46, wherein the CAR T-cell is a 1st generation CAR T-cell, a 2nd generation CAR T-cell, a 3rd generation CAR T-cell, a 4th generation CAR T-cell, a TRUCK, a smart CAR, or an iCAR.

47. The method according to claim 29, wherein the anti-CD25 ADC is ADCx25, ADCT 301, or Camidanlumab tesirine.

48. The method of claim 29, wherein the ADC is administered in combination with:
(i) a therapeutic agent;
(ii) a corticosteroid;
(iii) dexamethasone;
(iv) an IL-6 antagonist; and/or
(v) tocilizumab.
